(19) United States
(12) Patent Application Publication
(10) Pub. No.: US 2003/0077828 A1
(43) Pub. Date: Apr. 24, 2003

(54) Methods for highly efficient generation of adenoviral vectors

(76) Inventor: Yifan Dai, Grayslakes, IL (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/010,644

(22) Filed: Nov. 8, 2001

Related U.S. Application Data
(63) Continuation of application No. 08/789,886, filed on Jan. 28, 1997, now abandoned.

(51) Int. Cl. C12N 15/861; C07H 21/04; C12N 7/00

(52) U.S. Cl. 435/456; 435/235.1; 435/320.1; 536/23.1

(57) ABSTRACT

The invention provides reagents and methods for highly efficient generation of adenoviral vectors by homologous recombination. The present invention provides unique shuttle vectors and an improved methodology for co-transfection of a shuttle vector and a helper plasmid into 293 cells to generate E1-deleted, E1/E3-deleted, E1/E2a/E3-deleted or E1/E3/E4/protein IX-deleted adenoviral vectors.

### RELATED APPLICATIONS

This application is a continuation of U.S. patent application Ser. No. 08/789,886, filed Jan. 28, 1997.

### BACKGROUND OF THE INVENTION

1. Technical Field of the Invention

The invention provides reagents and methods for highly efficient generation of adenoviral vectors by homologous recombination. The present invention provides unique shuttle vectors and an improved methodology for co-transfection of a shuttle vector and a helper plasmid into 293 cells to generate E1-deleted, E1/E3-deleted, E1/E2a/E3-deleted or E1/E3/E4/protein IX-deleted adenoviral vectors.

2. Description of the Related Art

Adenoviruses (Ad) consist of nonenveloped icosahedral (20 facets and 12 vertices) protein capsids having a diameter of 60-90 nm and an inner DNA/protein core (1). The outer capsid is composed of 252 capsomers arranged geometrically to form 240 hexons (12 hexons per facet) and 12 penton bases; the latter are located at each vertex from which protrude as antennalike fibers. This structure is responsible for attachment of Ad to cells during infection. Wild-type Ad contain 87% protein and 13% DNA and have a density of 1.34 g/ml in CsCl. The adenoviral genome is a double-stranded linear DNA molecule of approximately 36 kb and is conventionally divided into 100 map units (mu). Each end of the viral genome is terminated by a region containing a 100-150 bp repeated DNA sequence, termed an inverted terminal repeat (ITR). The left ITR (bp 194-385) contains the signal for encapsidation (the “packaging signal”). Both ITRs and the packaging signal are cis-acting elements necessary for viral DNA replication and packaging (2, 3).

Ad vectors are utilized in the field of gene therapy because of several useful characteristics. Such characteristics include: (a) Ad have been widely studied and well characterized as a model system for eukaryotic gene regulation and, as such, have served as a basic tool for viral vector development; (b) Ad vectors are relatively simple to generate and manipulate; (c) Ad exhibit a broad host range in vitro and in vivo with high infectivity, and have the ability to infect non-dividing cells; (d) Ad are relatively stable and may be isolated at high titer [10¹⁰-10¹² plaque-forming unit (pfu)/ml]; (e) the life cycle of Ad does not require integration into the host cell genome; (f) the foreign genes delivered by Ad vectors are mainly expressed episomally, thus having low genotoxicity when applied in vivo; and, (g) side effects have not been reported following vaccination of volunteers with wild-type Ad, demonstrating their safety for in vivo gene transfer. Additionally, Ad vectors have been successfully utilized in studies of eukaryotic gene expression (11, 12), vaccine development (13, 14), and gene transfer in animal models (4, 15, 16). Experimental routes for administrating recombinant Ad to different tissues in vivo have included intratracheal instillation (17), intramuscular injection (18), peripheral intravenous injection (19), and stereotactic inoculation of the brain (20).

The Ad genome is further subdivided into early (E) and late (L) regions consisting of separate transcription units characterized according to the onset of viral DNA replication. The E1 region (E1A and E1B) encodes proteins responsible for the regulation of transcription of the viral genome as well as a few cellular genes (6). Expression of the E2 region genes (E2A and E2B) leads to the synthesis of the proteins needed for viral DNA replication (7). The proteins encoded by the E3 region prevent cytolysis by cytotoxic T cells and tumor necrosis factor (8). The E4 proteins (encoded by genes of the E4 region) are involved in DNA replication, late gene expression and splicing, and host cell shut off (9). The products of the late genes, including the majority of the viral capsid proteins, are expressed after processing of a 20-kb primary transcript driven by the major late promoter (MLP) (10). The MLP (located at 16.8 mu) is particularly efficient during the late phase of infection, and the mRNAs issued from this promoter possess a 5′ tripartite leader (TL) sequence, which enhances translation of those mRNAs.

The E3 region is dispensable from the Ad genome (i.e., not required for replication; see ref. 25), and the first generation Ad vectors are able to incorporate foreign DNA into the E1 and/or E3 region (5). In nature, the Ad particle is able to accommodate DNA to a maximum size of approximately 105% of the wild-type genome (26), corresponding to an additional 2 kb of DNA. Combined with the approximate 6.5 kb of DNA that is replaceable in the E1 and E3 regions using current Ad helper systems (see below), the maximum capacity of the current Ad vector for heterologous DNA is under 8.5 kb. This corresponds to approximately 15% of the total length of the vector. Replication may occur with the first generation Ad vectors at high multiplicities of infection (moi) since the replication deficiency rendered by the E1 deletion is incomplete (27). Leakage represents a source of vector-borne cytotoxicity in target cells and is responsible for the induction of inflammatory and immune responses to vector-infected target cells in vivo (28). These factors, at least in part, account for the transient nature of transgene expression using current Ad vectors.

The size limit on DNA that can be packaged into Ad virions is similar to the size of the wild-type Ad genome; the total capacity of an Ad virion has been determined to be approximately 5% greater than the wild-type Ad genome. Thus, insertion of large fragments of heterologous DNA into Ad requires replacement of viral sequences. In order to generate a viable, recombinant Ad vector including a heterologous DNA fragment (or “gene of interest”), the function of the replaced viral DNA must be either dispensable or supplied by a trans-acting source (a “helper” source). The trans-acting source may take the form of a helper virus and/or a helper cell. The current state of the art provides two types of Ad vector systems, a helper-dependent system and a helper-independent system.

Helper virus-dependent Ad were utilized originally by those skilled in the art to generate infectious, recombinant Ad vectors. Over time, this system has been developed into a vector system useful for delivering heterologous genes. For use in gene therapy, however, this vector system provides a significant difficulty to the investigator. Namely, the system leads to helper virus contamination of the recombinant Ad vector product. This problem is extremely relevant if the recombinant Ad vector is to be administered to a patient as part of a gene therapy protocol. Therefore, this approach has been largely abandoned in favor of the development of helper virus-independent systems. Recently, systems have been developed that include helper virus-independent and replication-defective vectors.

The development of a helper virus-independent Ad vector was dependent upon the development of an Ad “helper” cell line, 293 (ATCC# CRL 1573). The 293 cell line was derived from human embryo kidney cells by transformation with Ad5 DNA fragments. The transformed cell line constitutively expresses the E1 proteins (E1A and E1B), which are required for Ad replication (FIG. 2; see Ref. 2). The 293 cell line, therefore, provides the E1 proteins in trans, such that an adenoviral vector having a deletion in the E1 region may replicate within 293.

Three major approaches have been utilized by those skilled in the art in combination with the 293 cell line to generate an infectious, replication-defective helper virus-independent recombinant Ad vector. One approach, commonly referred to as the Stow method (22), is an in vitro recombination approach, involving transfection of 293 cells with a modified Ad genome including a gene of interest inserted in the E1 region. The method requires isolation of an Ad genome and restriction enzyme digest of the genome such that a small and a large fragment representing a portion of the left end and the remaining left end and the right end, respectively, of the Ad genome are isolated. A gene of interest is then inserted into the E1 region of the small fragment. This modified small fragment is then ligated to the large fragment and the recombined molecule transfected into 293 cells (FIG. 3A). This method requires the investigator to perform multiple steps and is not convenient for routine generation of recombinant Ad vectors.

A second approach requires isolation of a small fragment and a large fragment of the Ad genome following restriction enzyme digestion of the Ad genome. Into the E1 region of the small fragment, consisting of a portion of the left end of the Ad genome, is inserted a gene of interest. The recombined DNA molecule is then circularized to form a plasmid. The isolated large fragment of the Ad genome (having some overlapping sequence with the small fragment) and the recombinant plasmid are then co-transfected into 293 cells. To generate recombinant Ad vectors using this system, recombination between the large fragment and the recombinant plasmid must occur within the 293 cell (“in vivo recombination”; ref. 23). This method requires an extensive amount of labor by the investigator (i.e., isolate the Ad genome, restriction digest and isolate the small and large fragments, clone in a gene of interest, and is, therefore, inconvenient as a method for the routine generation of recombinant Ad vectors.

A third method requires recombination between two plasmids within a cell (24). One of these plasmids (the “shuttle vector”) includes a portion of the left end of the Ad genome having a gene of interest inserted into the E1 or E3 region. The other plasmid (the “helper plasmid”) is a recombinant plasmid having the remainder of the Ad genome (some of which overlaps with the shuttle vector) and plasmid backbone sequence. These two plasmids are co-transfected into 293 cells resulting in the generation of recombinant Ad, provided recombination between the two plasmids occurs within a 293 cell (FIG. 3B). Such a system has been developed by Frank Graham and is the method of recombinant Ad vector generation most commonly utilized by those skilled in the art (5, 29, 30). In that system, four separate Ad type 5 (Ad5) helper plasmids may be utilized depending on the specific application: 1.) pJM17 (29; available from Microbix Biosystems, Inc., Ontario, Canada); 2.) pBHG10 (30; available from Microbix Biosystems, Inc., Ontario, Canada); 3.) pBHG11 (30; available from Microbix Biosystems, Inc., Ontario, Canada); or, 4.) pKGB1(32).

pJM17 has been shown to be useful for generating E1-deleted Ad vectors, although the capacity for exogenous DNA is only approximately 4.7-4.9 kb. Additionally, use of pJM17 often results in the generation of large amounts of wild-type virus when the pBR322 sequence in the Ad5 genome is deleted. These represent significant limitations to the as widespread use of the pJM17 helper plasmid in gene therapy or basic research.

These problems have been partially, but not completely, overcome through the use of the pBHG10 and pBHG11 helper plasmids, which have more capacity (from 7.8 kb to 8.3 kb) for exogenous genetic material. Also, use of pBHG10 and pBHG11 do not result in a wild-type viral particle production due to deletion of the Ad5 packaging signal (30). pBHG10 and pBHG11 have been shown to be useful in the generation of E1/E3-deleted Ad vectors having a total capacity for 7.9 to 8.3 kb of exogenous DNA (30). Additionally, pKGB1 has been utilized for generating E1/E3/E4/protein IX-deleted Ad vectors having a total capacity of 11 kb for exogenous DNA (32).

Graham's system also includes three shuttle vectors into which the gene of interest may be inserted into the E1 region: 1.) pΔE1p1A (30; available from Microbix Biosystems, Inc., Ontario, Canada); 2.) pΔE1p1B (30; available from Microbix Biosystems, Inc., Ontario, Canada); and, 3.) pXCJL1 (a derivative of pXCX2; 31). Each of these shuttle vectors comprise the Ad5 left ITR including the packaging signal (ad5 sequence bp 22 to 342 for pΔE1p1A and pΔE1p1B; ad5 sequence bp 22-450 for pXCJL1), a homologous recombination arm (ad5 sequence bp 3524 to 5790 bp for pΔE1p1A and pΔE1p1B; ad5 sequence bp 3332-5788 bp for pXCJL1), and a multiple cloning site between the ad5 packaging signal and the homologous recombination arm into which exogenous DNA may be inserted (30). The shuttle vector comprising the gene of interest (i.e., the CMV-EGFP cassette as shown in FIG. 4) is typically co-transfected with one of the helper Ad5 plasmids (i.e., pBHG10 as in FIG. 4) into early passage 293 cells. Homologous recombination between the helper plasmid and the shuttle vector results in the generation of a recombinant Ad vector (FIG. 4).

Graham's method is more convenient to the investigator than either the first or the second methods described above, but maintains at least two significant drawbacks. For instance, the frequency of recombination between the plasmids is, in general, very low. The efficiency of generating a recombinant Ad vector generation using pBHG10 or pBHG11 is usually low and the system typically requires multiple co-transfection in order to generate recombinant Ad vector. An additional limitation is that Graham's methods require the use of early passage 293 cells (earlier than passage 40-50) (5). Accordingly, there is a need in the art for improved methods for efficient generation of recombinant Ad vectors. The present invention provides a significant improvement over this third method.

### SUMMARY OF THE INVENTION

The reagents and methodologies provided in this invention allow for generation of recombinant Ad vectors with much higher efficiency than conventional methods. A significant difficulty encountered by investigators in the field of Ad research and gene therapy, efficient generation of recombinant Ad vectors, may be overcome using the reagents and methodologies of the present invention.

An objective of the present invention is to provide reagents for high-efficiency generation of a recombinant Ad vector. The present invention thus includes several unique shuttle vectors comprising: 1). an ad5 3′-ITR/5′-ITR packaging signal fusion structure (3′ITR: ad5 bp 35503 to 35925; 5′ITR/packaging signal: ad5 bp 4 to 450); 2). a polylinker region; and, 3). a region comprising ad5 sequence capable of functioning as a homologous recombination region, preferably comprising Ad5 bp 3332-5578 or Ad5 bp 3533-5788.

Another objective of the present invention is to provide methodologies for high-efficiency generation of a recombinant Ad vector. The present invention, therefore, includes a method comprising pre-incubation of a shuttle vector and an Ad5 helper plasmid at increased temperature for a sufficient period of time, preferably at 70° C. for 5 min in a conditional buffer.

A further objective of the present invention is to provide a method for enhancing the efficiency of Ad vector production through the use of late passage 293 cells, preferably at passage 70 or later.

Yet another objective of the present invention is to provide reagents and methods useful for high-efficiency generation of E1-deleted, E1/E3-deleted, E1/E2a/E3-deleted or E1/E3/E4/protein IX-deleted adenoviral vectors.

The objectives described above, as well as other objectives of the present invention, will be understood in light of the detailed description of the invention provided below.

### BRIEF DESCRIPTION OF THE FIGURES

FIG. 1. The genome and transcription units of Ad5. The length of the Ad5 genome is approximately 36 kb, and is conventionally divided into 100 map units (mu). Early (E) region and late (L) region genes are indicated, and refer to the time period during which these genes are transcribed following infection of a host cell. The orientation of transcription is indicated by arrows. Gaps between arrows indicate intervening sequences. The box represents the location of the major late promoter (MLP). The triangle at map unit 1 represents the location of the packaging signal (referred to in the text as Ψ).

FIG. 2. Development of the 293 cell line. A human embryonic kidney cell was transformed with fragments of the Ad5 genome. The cell expresses the Ad E1 proteins.

FIG. 3. Methods for generating recombinant Ad vectors. A. The Stow method, an in vitro recombination system. B. The plasmid recombination method.

FIG. 4. Method for generating a recombinant Ad vector. The shuttle vector GT4122 comprising a CMV-EGFP insert was co-transfected with pBHG10 into 293 cells. Homologous recombination between the helper plasmid and the shuttle vector results in generation of a recombinant Ad5 CMV-EGFP vector.

FIG. 5. Recombination between shuttle vector GT4122 and pBHG10. In 293 cells, pBHG10 replicates in the presence of E1 protein. Shuttle vector GT4122 does not self-replicate, reducing the probability of homologous recombination.

FIG. 6. Recombination between shuttle vector GT4117 and pBHG10. In 293 cells, pBHG10 replicates in the presence of the E1 protein. Shuttle vector GT4117, comprising both a 5′ and a 3′ ITR also replicates as a mini virus, which increases the probability of homologous recombination.

FIG. 7. Maps of GT4120, GT4121 and GT4142. GT4120 comprises an Ad5 5′-ITR/packaging signal (Ψ) structure. GT4121 and GT4142 each comprise an Ad5 3′-ITR and a 5′-ITR/packaging signal (Ψ) fusion structure.

FIG. 8. Maps of GT4122 and GT4117. GT4122 comprises an Ad5 5′-ITR/packaging signal (Ψ) structure. GT4117 comprises an Ad5 3′-ITR and a 5′-ITR/packaging signal (Ψ) fusion structure. Each plasmid further comprises a CMV-EGFP cassette and additional Ad5 sequence (bp 3533 to 5788 of the Ad5 genome).

FIG. 9. Maps of GT4140 and GT4141. GT4140 contains ad5 5′-ITR/packaging signal (Ψ) structure. GT4141 comprises an Ad5 3′-ITR and a 5′-ITR/packaging signal (Ψ) fusion structure. Each plasmid further comprises a CMV-EGFP cassette and additional Ad5 sequence (bp 3332 to 5788 of the Ad5 genome).

FIG. 10. Ad vector generation using late-passage 293 cells. One μg of GT4122 or GT4117 was combined with two μg pBHG10 and co-transfected into 293 cells (passage 71) in one well of a 6-well plate and split into two 24-well plates (48 wells total) two days later. Green fluorescent plaques were counted on day 15 after transfection.

FIG. 11. Other improvements of Ad vector shuttle vectors. Plasmid A comprises an Ad5 3′-ITR and a 5′-ITR/packaging signal (Ψ) fusion structure in opposite orientation. The ITRs may also be separated by a DNA sequence. Plasmid B comprises a 5′-ITR with or without packaging signal (Ψ) in reverse orientation and a 5′-ITR with packaging signal in a forward orientation.

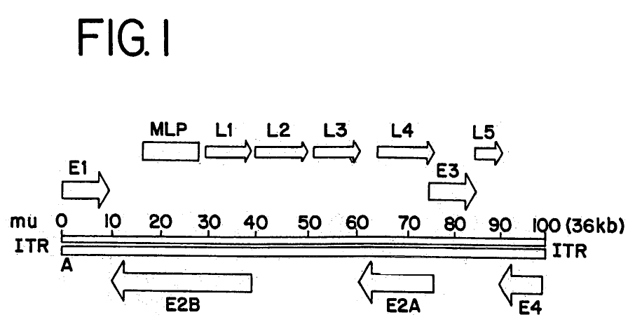

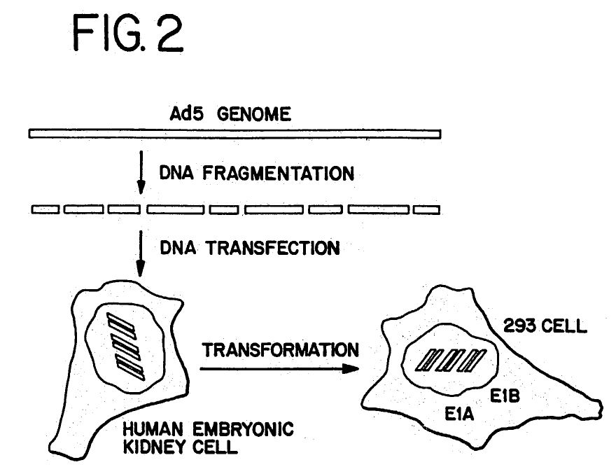

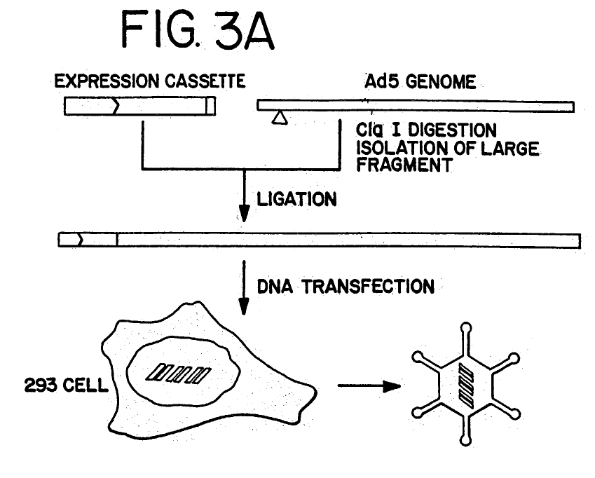

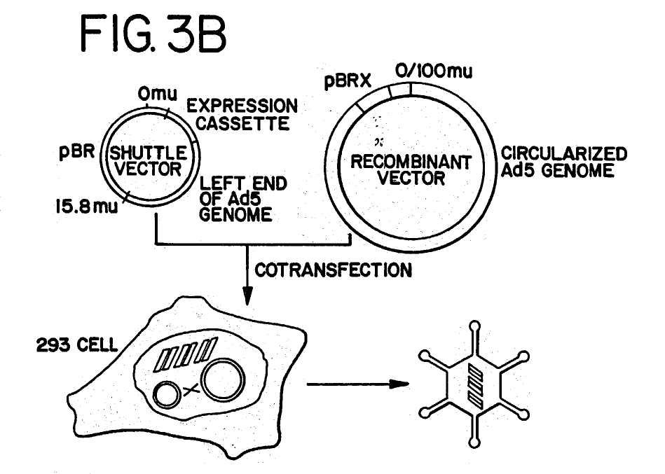

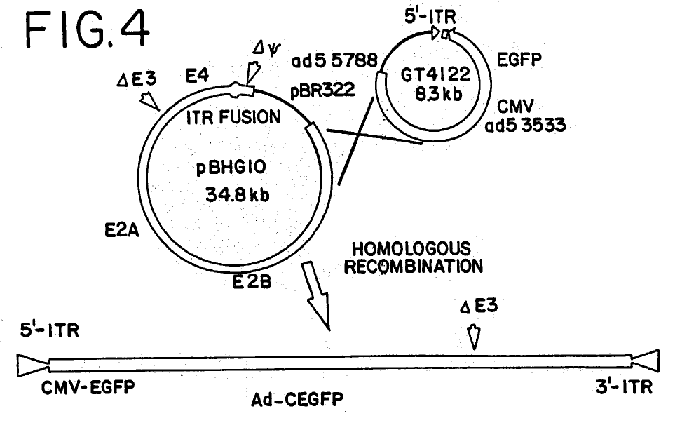

### DETAILED DESCRIPTION OF THE INVENTION

Within this application, unless otherwise stated, the techniques utilized may be found in any of several well-known references including: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), and Manipulation of adenovirus vectors. (In Methods in Molecular Biology (Vol. 7), Gene Transfer and Expression Protocols, pp. 109-128, ed. E. J. Murray, The Humana Press Inc., Clifton, N.J.).

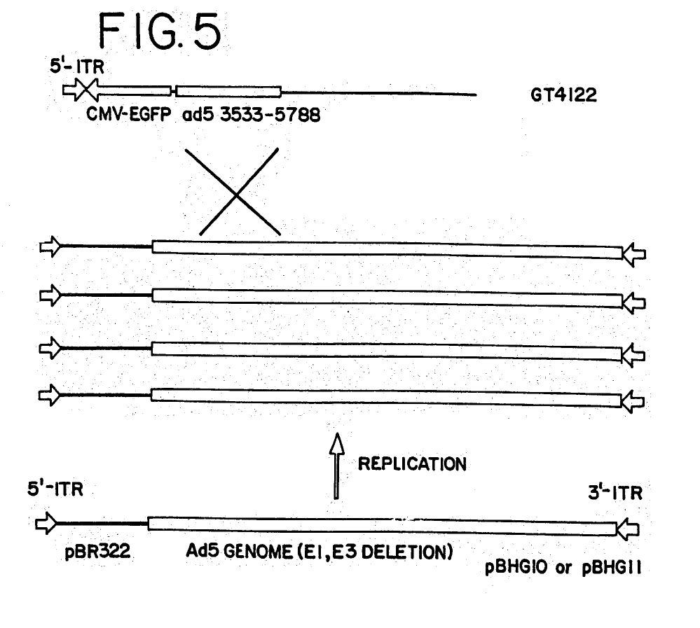

The present invention provides reagents and methods useful for increasing the efficiency of recombinant Ad vector production. This invention provides a shuttle vector that allows for increased efficiency in generation of Ad vectors by homologous recombination. The three conventional shuttle vectors described above (pΔE1p1A, pΔE1p1B and pXLJL1) comprise only the 5′-ITR comprising the packaging signal of Ad5 and are unable to replicate within a host cell (FIG. 5). Therefore, following co-transfection into a host cell, the probability that the shuttle vector and the helper plasmid will recombine to generate a recombinant Ad vector is limited by the copy number of the shuttle vector. Accordingly, recombination events are infrequent and the generation of a recombinant Ad vector is rare using these conventional reagents.

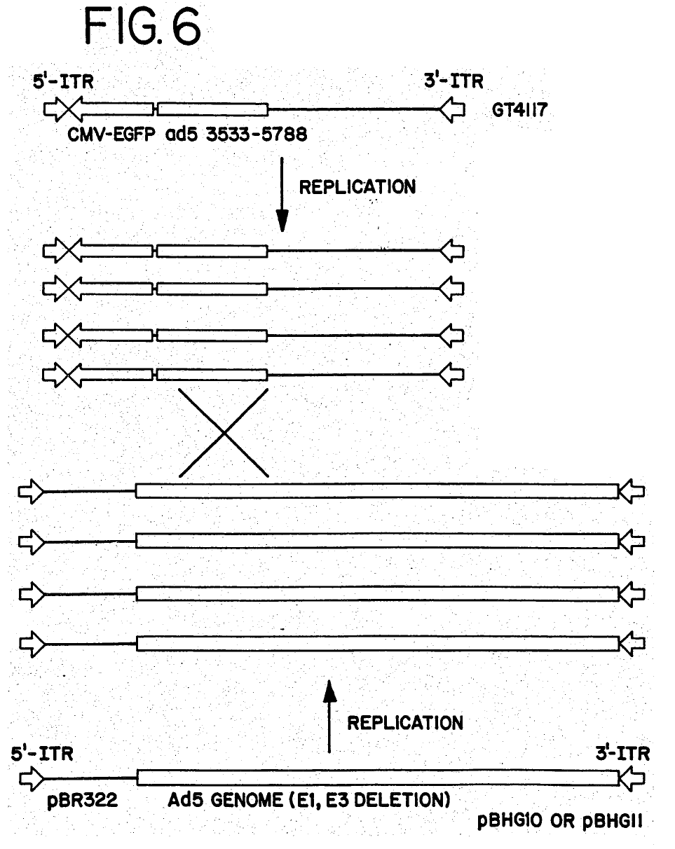

The present invention provides multiple shuttle vectors comprising both a 5′-ITR with the packaging signal (Ψ) and a 3′-ITR that are able to replicate within a host cell. Following co-transfection with a helper plasmid into 293 cells (ATCC# CRL1573), a shuttle vector of the present invention is able to replicate and increase its copy number. This results in an increased possibility of homologous recombination (due to an increased number of available shuttle vectors) between the shuttle vector and the helper plasmid (FIG. 6). Accordingly, the efficiency of recombinant Ad vector generation using a shuttle vector of the present invention is greatly increased over that achieved using a conventional shuttle vector.

Preferably, exogenous DNA comprising a gene of interest may be incorporated into a shuttle vector of the present invention. The gene of interest may be a reporter or an effector gene. Useful reporter genes may include but are not limited to β-galactosidase (β-gal), luciferase, and, as demonstrated within this application, green fluorescent protein (GFP). Useful effector genes may include but are not limited to a gene encoding an antigen, a tumor suppressor gene, a growth suppressor gene, an oncogene, an immunomodulatory gene or a ribozyme. The gene of interest may further include a regulatory element operably linked to a reporter or an effector gene such that expression is controllable or limited to specific tissues resulting in a higher therapeutic efficacy of the vector. The Ad vector system of the present invention is also useful for accelerating gene transfer and gene therapy research by providing a more efficient method for generating recombinant Ad vectors. This system may also be useful in basic research of the Ad life cycle and the mechanisms with which Ad may infect and thrive within a host cell.

The reagents and/or methodologies of the present invention may be combined in various combinations to supply a kit for recombinant adenoviral vector production. Such a kit may include a shuttle vector of the present invention (i.e., GT4117, GT4121, GT4142, or GT4141), a helper plasmid (i.e., pJM17, PBHG10, or pBHG11), and a cell line capable of supplying in trans proteins required for adenoviral replication (i.e., 293 cells). The shuttle vector of the kit may be supplied having a reporter gene (i.e., β-gal) or effector gene (i.e., IFN-γ) incorporated into its structure. Alternatively, the shuttle vector may be provided that has a polylinker region such that the investigator may insert a gene of interest specific for their particular application.

The present invention further provides a method for increasing the efficiency of recombinant Ad vector generation using either conventional reagents or the improved reagents of the present invention. The present invention provides a unique methodology comprising novel conditions for incubation of a shuttle vector and a helper plasmid prior to co-transfection that results in high-efficiency generation of recombinant Ad vectors. Using conventional methods, the shuttle vector and the helper plasmid are incubated at room temperature prior to transfection into 293 cells (5). In both conventional and the inventive methodology of the present application, the 293 cells may be transfected using any of the known transfection protocols know to one skilled in the art such as calcium phosphate-mediated transfection (i.e., using a kit available from GIBCO/BRL), lipofection (i.e., Lipofectamine available from GIBCO/BRL), or electroporation (i.e., using technology availabe from BioRad, Inc.). For the purposes of this invention, the initial incubation of shuttle vector and helper plasmid occurs under higher temperatures than that at which conventional incubations are performed. The temperature is higher than room temperature, preferably in the range of 35° C. to 80° C., more preferably being 50° C. to 80° C., and most preferably being 70° C. This new methodology results in the generation of a higher number of recombinant Ad vectors per transfection, allowing the investigator to avoid time-consuming, multiple, repeated transfections associated with conventional methodologies.

Additionally, a methodology for high-efficiency generation of recombinant Ad vectors is provided using “late passage” 293 cells. The late passage 293 cells have preferably been passaged greater than 50 times, and even more preferably greater than or equal to 70 times. This is convenient for the investigator, in that he may utilize a single batch of cells for a much longer period of time, avoiding the time-consuming task of repeated initialization of 293 cell culture.

Another particularly useful embodiment of the present invention combines both increased temperature and late passage 293 cells, resulting in both increased efficiency of recombinant Ad vector generation and ease of use for the investigator.

Within this application, a DNA fragment is defined as segment of a single- or double-stranded DNA derived from any source.

A DNA construct is defined a plasmid, virus, autonomously replicating sequence, phage or linear segment of a single- or double-stranded DNA or RNA derived from any source.

A reporter gene is defined as a subchromosomal and purified DNA molecule comprising a gene encoding an assayable product.

An assayable product includes any product encoded by a gene that is detectable using an assay. Furthermore, the detection and quantitation of the assayable product is anticipated to be directly proportional to the level of expression of the gene.

An effector gene is defined as any gene that, upon expression of the polypeptide encoded by the gene, confers an effect on an organism, tissue or cell.

Heterologous DNA or exogenous DNA is defined as DNA introduced into an adenoviral construct that was isolated from a source other than an adenoviral genome.

A transgene is defined as a gene that has been inserted into the genome of an organism other than that normally present in the genome of the organism.

A recombinant adenoviral (Ad) vector is defined as a adenovirus having at least one segment of heterologous DNA included in its genome.

Adenoviral (Ad) particle is defined as an infectious adenovirus, including both wild type or recombinant. The adenovirus includes but is not limited to a DNA molecule encapsidated by a protein coat encoded within an adenoviral genome.

A recombinant adenoviral (Ad) particle is defined as an infectious adenovirus having at least one portion of its genome derived from at least one other source, including both adenoviral genetic material as well as genetic material other than adenoviral genetic material.

An antigen is defined as a molecule to which an antibody binds and may further inlcude any molecule capable of stimulating an immune response, including both activation and repression or suppression of an immune response.

A tumor suppressor gene is defined as a gene that, upon expression of its protein product, serves to suppress the development of a tumor including but not limited to growth suppression or induction of cell death.

A growth suppressor gene is defined as a gene that, upon expression of its protein product, serves to suppress the growth of a cell.

An oncogene is defined as a cancer-causing gene.

An immunomodulatory gene is defined as any gene that, upon expression of its nucleic acid or protein product, serves to alter an immune reaction.

A ribozyme is defined as an RNA molecule that has the ability to degrade other nucleic acid molecules.

The following examples illustrate particular embodiments of the present invention and are not limiting of the specification and claims in any way.

### EXAMPLE 1

### Construction of New Shuttle Vectors

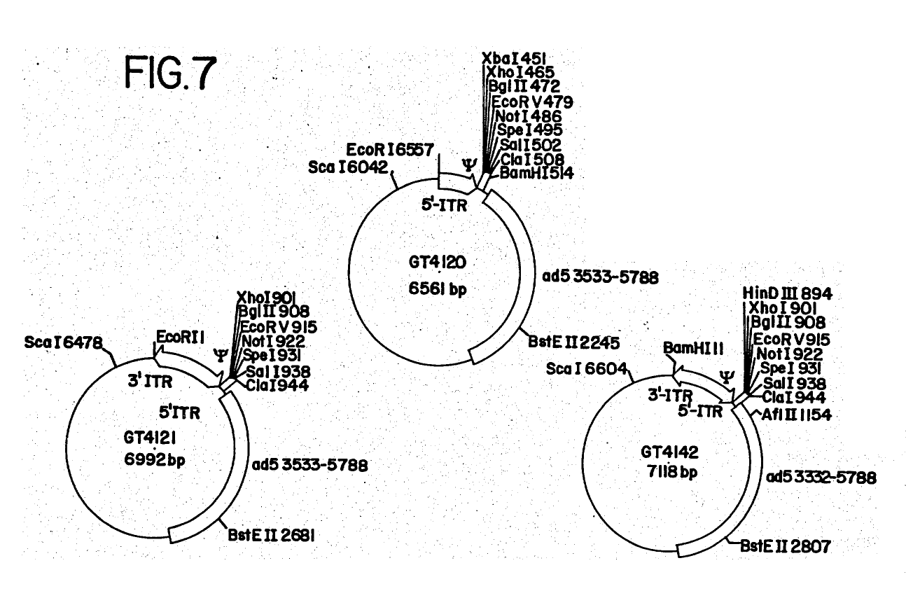

GT4120 is a conventionally utilized shuttle vector and comprises an Ad5 5′-ITR/packaging signal fusion structure (ad5 bp 22 to 450), a polylinker region and 2255 bp of additional Ad5 sequence (bp 3533 to 5788 of the Ad5 genome) in a pBR322 backbone (3.7 kb of pBR322 corresponding to the Sal I to EcoRI fragment). This plasmid was constructed by replacement of the Xba I to Afl II region of pXCJL1 (a derivative of pXCX2, see Ref. 31) with a polylinker sequence at Xba I/Bam HI (FIG. 7). A shuttle vector of the present invention, GT4121, was constructed by replacement of the Ad5 5′-ITR/packaging signal structure of GT4120 with the Ad5 3′-ITR/5′-ITR/packaging signal fusion structure (comprising Ad5 bp 35503-35925 and Ad5 bp 4-450) from GT4007 (FIG. 7). Another shuttle vector of the present invention, GT 4142, is identical to GT4121 except that GT4142 comprises additional Ad5 sequence (Ad5 bp 3332-5788) as a homologous recombination arm (FIG. 7C).

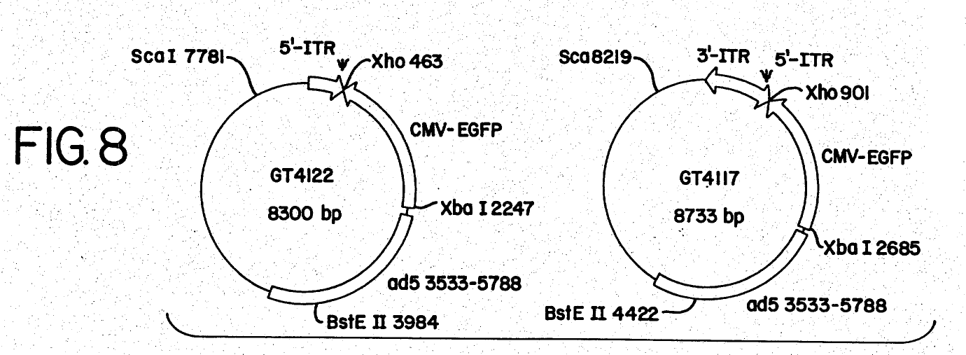

The conventionally utilized shuttle vector, GT4122, was constructed by insertion of the CMV-EGFP expression cassette of plasmid GT4082 (available from Baxter Healthcare Corp., Round Lake, Ill.) into the conventionally utilized plasmid GT4120. GT4122 comprises an Ad5 5′-ITR/packaging signal structure (bp 22-450), a CMV-EGFP expression cassette in reverse orientation, additional Ad5 sequence (bp 3533-5788) and a portion of the pBR322 backbone (Sal I/EcoR I fragment) (FIG. 8). To construct the inventive plasmid GT4117, the 5′-ITR of plasmid GT4122 was replaced by the Ad5 3′-ITR/5′-ITR/packaging signal fusion structure (comprising Ad5 bp 35503-35925 and Ad5 bp 4-450) (FIG. 8).

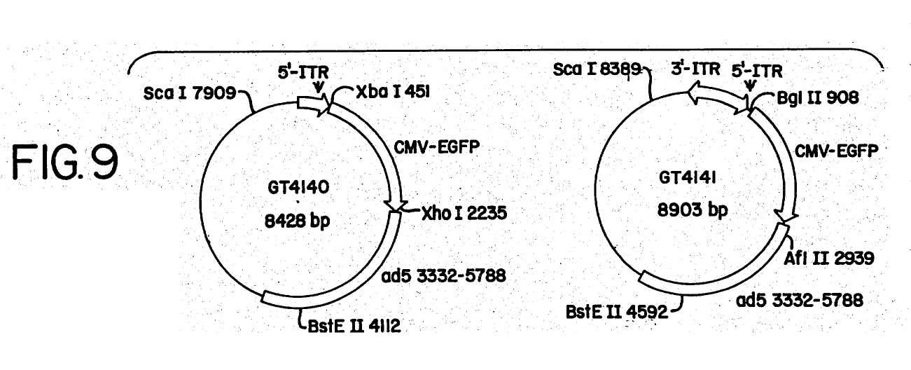

The conventionally utilized plasmid GT4140 was constructed by insertion of a CMV-EGFP expression cassette isolated from GT4082 into the XhoI/XbaI site of pXCJL1. GT4140 comprises an Ad5 5′-ITR/packaging signal (bp 22-450) structure, a CMV-EGFP expression cassette in forward orientation, additional Ad5 sequence (bp 3332-5788) and a portion of pBR322 (the Sal I/EcoR I fragment) as plasmid backbone (FIG. 9). The inventive plasmid GT4141 was constructed by insertion of the CMV-EGFP expression cassette taken of GT4082 into the XhoI/SpeI site of the inventive plasmid GT4142. GT4141 comprises an Ad5 3′-ITR/5′-ITR/packaging signal fusion structure (Ad5 bp 35503-35925 and ad5 bp 4-450), a CMV-EGFP expression cassette in forward orientation, additional Ad5 sequence (bp 3332-bp 5788) and a portion of pBR322 (the SalI EcoRI fragment) as plasmid backbone (FIG. 9).

### EXAMPLE 2

### Generation of a Recombinant Ad Vector (Adcmv-EGFP) Using Invention Shuttle Vectors

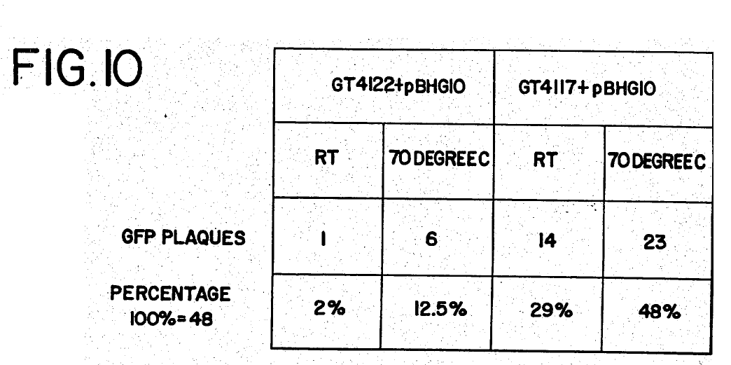

In order to determine the increased efficacy of Ad vector generation using the reagents of the present invention, conventional techniques were utilized to co-transfect 293 cells with the shuttle vector of the invention with pBHG10 or pBHG11. In this example, late-passage 293 cells were utilized (passage 71), were utilized. It is to be understood by one skilled in the art that the invention plasmids may be utilized to transfect lower-passage 293 cells (under passage 50). At room temperature, one μg of GT4122 or GT4117 was independently mixed with two μg of pBHG10 in 20 μl of H₂O for five min. This was followed by mixing with 25 μl of 2.5 M CaCl₂ and 205 μl of H₂O, and mixing with 250 μl 2×BBS (50 mM BES pH 6.95, 280 mM NaCl, 1.5 mM NaH₂PO₄), and incubation at room temperature for 10 min. The mixture was then added to 293 cells in one well of a 6-well plate with 4 ml of growth medium (10% new born calf serum+90% DMEM). The transfected 293 cells were then incubated in a 37° C., 3% CO₂ incubator overnight (12-15 hrs). The media was changed next day and the cells were incubated in a 37° C., 5% CO₂ incubator for another 24 hr. Cells from one well of 6-well plate were then split into two 24-well plates and incubated in a 37° C., 5% CO₂ incubator for 2-3 weeks. Medium was changed every 3-4 days during this period. Plaques of recombinant viral particles comprising the CMV-EGFP expression cassette were detected and counted under a green fluorescence microscope. Utilization of the invention shuttle vector GT4117/pBHG10 combination generated 14 times more green fluorescent plaques than utilization of the conventional shuttle vector GT4122/pBHG10 combination (FIG. 10).

### EXAMPLE 3

### Improved Methodology for Recombinant Adenoviral Vector Production

The present invention also includes a methodology with which the efficiency of Ad vector production is increased over conventional methods. This methodology includes incubation of shuttle vector and helper plasmid at temperatures above that utilized in conventional methodologies. The improved methodology may be utilized with either conventional shuttle vector or using the improved shuttle vectors of the present invention. Additionally, the methodology of this example may be utilized with either late-passage or early-passage (less than passage 50) 293 cells.

In this example of the present invention, the initial incubation of the shuttle and helper plasmids is performed at a temperature greater than room temperature (the conventional methodology). The methodology of the present invention, utilized in this example, was performed as follows: one μg of GT4122 or GT4117 was mixed with two μg of pBHG10 in 18 μl of H₂O and two μl of 10×conditional buffer (500 mM Tris-HCl, pH 7.5, 330 mM NaCl, 110 mM MgCl₂) was added, followed by incubation at 70° C. for 5 min. The mixture was then cooled at room temperature and mixed with 25 μl of 2.5 M CaCl₂ and 205 μl of H₂O, followed by mixing with 250 μl 2×BBS (50 mM BES pH 6.95, 280 mM NaCl, 1.5 mM NaH₂PO₄), followed by incubation at room temperature for 10 min. The mixture was then added to 293 cells in one well of a 6-well plate with 4 ml of growth medium (10% new born calf serum+90% DMEM). The transfected 293 cells were then incubated in a 37° C., 3% CO₂ incubator overnight (12-15 hrs). The media was changed next day and cells were incubated in a 37° C., 5% CO₂ incubator for another 24 hr. Cells from one well of 6-well plate were then split into two 24-well plates and incubated in a 37° C., 5% CO₂ incubator for 2-3 weeks. Medium was changed every 3-4 days during this period. Plaques of recombinant viral particles comprising the CMV-EGFP expression cassette were detected and counted under a green fluorescence microscope.

FIG. 10 demonstrates the results of co-transfection of late-passage 293 cells (passage 71) with the shuttle vectors of the present invention with conventional helper plasmids. Following an initial 70° C. incubation of either the invention shuttle vector GT4117 or the conventional shuttle vector GT4122 with the helper plasmid pBHG10, co-transfection of 293 cells with the GT4117/pBHG10 mixture results in the detection of four times more green fluorescent plaques than co-transfection of 293 cells with the GT4122/pBHG10 mixture. In both the GT4122/pBHG10 and the GT4117/pBHG10 co-transfection groups, initial incubation of shuttle vector and helper plasmid at 70° C. generates a greater number of fluorescent plaques than those groups initially incubated at room temperature (FIG. 10).

In summary, the shuttle vector of the present invention combined with an initial incubation of the shuttle vector and the helper plasmid at 70° C. increase the efficiency of recombinant Ad vector generation by greater than 20-fold as compared to that achieved using conventional techniques including a shuttle vector comprising only a single ITR and an initial incubation of shuttle vector and helper plasmid at room temperature.

### EXAMPLE 4

### Other Designs of Improved Shuttle Vector

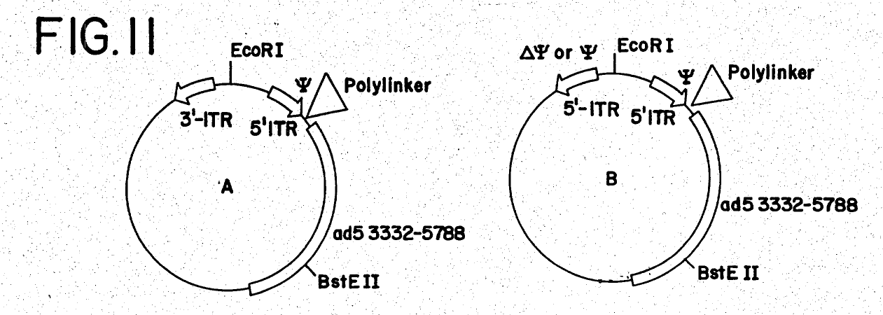

It is also possible to separately incorporate two ITR regions into a shuttle vector. FIG. 11 shows two new designs of new shuttle vectors of this invention. Plasmid A comprises both a 3′-ITR in reverse orientation, an intervening DNA sequence and a 5′-ITR/packaging signal fusion structure in the forward orientation, a polylinker region, and additional Ad5 sequence (bp 3332-5788 of the Ad genome) as a homologous recombination arm. Plasmid B comprises a 5′-ITR (with or without packaging signal) in reverse orientation and a 5′-ITR having the packaging signal in the forward orientation, a polylinker region and additional Ad5 sequence (bp 3332-5788 of the Ad genome) as a homologous recombination arm (FIG. 11). Utilization of either shuttle vector results in the generation of E1-deleted, E1/E3-deleted, E1/E2a/E3-deleted or E1/E3/E4/protein IX-deleted recombinant Ad vectors with similar efficiency to GT4121 and GT4142.

While a preferred form of the invention has been shown in the drawings and described, since variations in the preferred form will be apparent to those skilled in the art, the invention should not be construed as limited to the specific form shown and described, but instead is as set forth in the claims.

### REFERENCES

1. Horwitz, M. S. 1990. Adenoviridae and their replication. In: Fundamental Virology (2nd Edn), eds. B. N. Field, D. M. Knipe, R. M. Chanock, J. L. Melnick, M. S. Hirsch, T. P Monath, B. Roizman. pp. 771-813. Raven Press, NY.

2. Sussenbach, J. S. 1984. The structure of the genome. In: The Adenoviruses. ed. H. S. Ginsberg, pp. 35-124. Plenum Publishing Corp., NY.

3. Philipson, L. 1984. Adenovirus assembly. In: The Adenoviruses. ed. H. S. Ginsberg, pp. 309-337. Plenum Publishing Corp., NY.

4. Stratford-Perricaudet, L., and M. Perricaudet. 1991. Gene transfer into animals: the promise of adenovirus. p. 51-61, In: Human Gene Transfer, Eds, O. Cohen-Haguenauer and M. Boiron, Editions John Libbey Eurotext, France.

5. Graham, F. L., and L. Prevec. 1991. Manipulation of adenovirus vectors. In: Methods in Molecular Biology (Vol. 7), Gene Transfer and Expression Protocols, pp. 109-128, ed. E. J. Murray, The Humana Press Inc., Clifton, N.J.

6. Nevins, J. R. 1993. Transcriptional activation by the adenovirus E1A proteins. Semin. Viol. 4: 25-31.

7. Pettersson, U., and R. J. Roberts. 1986. Adenovirus gene expression and replication: a historical review. In: Cancer Cells (Vol. 4): DNA Tumor Viruses. M. Botchan, T. Glodzicker, P. A. Sharp. Eds. pp. 37-57. Cold Spring Harbor Laboratory, Cold Spring Harbor, N.Y.

8. Wold, W. S. M., and L. R. Gooding. 1991. Region E3 of adenovirus: a cassette of genes involved in host immunosurveillance and virus-cell interactions. Virology 184: 1-8.

9. Halbert, D. N., J. R. Cutt, and T. Shenk. 1985. Adenovirus early region 4 encodes functions required for efficient DNA replication, late gene expression, and host cell shutoff. J. Virol. 56: 250-257.

10. Shaw, A. R., and E. B. Ziff. 1980. Transcriptions from the adenovirus-2 major later promoter yield a single early family of 3′ coterminal mRNA and five late families. Cell 22: 905-916.

11. Levrero, M., V. Barban, S. Manteca, A. Ballay, C. Balsamo, M. L. Avantaggiati, G. Natoli, H. Skellekens, P. Tiollais, and M. Perricaudet. 1991. Defective and nondefective adenovirus vectors for expressing foreign genes in vitro and in vivo. Gene 101: 195-202.

12. Ghosh-Choudhury, G., Y. Haj-Ahmad, P. Brinkley, J. Rudy, and F. L. Graham, F. L. (1986) gene 50, 161-171.

13. Grunhaus, A., and M. S. Horwitz. 1992. Adenoviruses as cloning vectors. Semin. Virol. 3:237-252.

14. Graham, F. L., and L. Prevec (1992) Adenovirus-based expression vectors and recombinant vaccines. in Vaccines: New Approaches to Immunological Problems, (Ellis, R. V. Ed.), pp. 363-390. Butterworth-heinemann, Buston.

15. Stratford-Perricaudet, L., L. Makeh, M. Perricaudet, and P. Briand (1992) Widespread long-term gene transfer to mouse skeletal muscles and heart. J. Clin. Invest. 90, 626-630.

16. Rich, D. P., M. Couture, L. M. Cardoza, V. M. Guiggio, D. Armentano, P. C. Espino, K. Hehir, M. J. Welsh, A. E. Smith, and R. J. Gregory (1993) Development and analysis of recombinant adenoviruses for gene therapy of cystic fibrosis. Human Gene Ther. 4, 461-476.

17. Rosenfeld, M. A., K. Yoshimura, B. C. Trapnell, K. Yoneyama, E. R. Rosenthal, W. Dalemans, M. Fukayama, J. Bargon, L. E. Stier, L. Stratford-Perricaudet, M. Perricaudet, W. B. Guggino, A. Pavirani, J.-P. Lecocq, and R. G. Crystal (1992) In vivo transfer of the human cystic fibrosis transmembrane conductance regulator gene to the airway epithelium. Cell 68, 143-155.

18. Quantin, B., L. D. Perricaudet, S. Tajbakhsh, and J.-L. Mandel. (1992) Adenovirus as an expression vector in muscle cells in vivo. Proc. Natl. Acad. Sci. USA 89, 2581-2584.

19. Herz, J. and R. D. Gerard (1993) Adenovirus-mediated transfer of low density lipoprotein receptor gene acutely accelerates cholesterol clearance in normal mice. Proc. Natl. Acad. Sci. USA 90, 2812-2816.

20. Le Gal La Salle, G., J. J. Robert, S. Bernard, V. Ridoux, L. D. Stratford-Perricaudet, and M. Perricaudet (1993) An adenovirus vector for gene transfer into neurons and glia in the brain. Science 259, 988-990.

21. Graham, F. L., J. Smiley, W. C. Russell, and R. Naim. 1977. Characteristics of a human cell line transformed by DNA from human adenovirus type 5. J. Gen. Virol. 36: 59-72.

22. Stow, N. D. 1981. Cloning of a DNA fragment from the bleft-hand terminus of the adenoviruse type 2 genome and its use in site-directed mutagenesis J. Virol. 37, 171-180.

23. Chinnadurai, G., S. Chinnadurai, and J. Brussa (1979) J. Virol. 32, 623.

24. Berkner, K. L. and P. A. Sharp (1983) Nucleic Acids Res. 17, 1983.

25. Jones, N., and T. Shenk. 1978. Isolation of deletion and substitution mutants of adenovirus type 5. Cell 13: 181-188.

26. Ghosh-Choudhury, G., Y. Haj-Ahmad, and F. L. Graham. 1987. Protein IX, a minor component of the human adenovirus capsid, is essential for the packaging of full-length genomes. EMBO J. 6: 1733-1739.

27. Jones, N., and T. Shenk. 1979. Isolation of adenovirus type 5 host range deletion mutants defective for transformation of rat embryo cells. Cell 17: 683-689.

28. Simon, R. H., J. F. Engelhardt, Y. Yang, M. Zepeda, S. Weber-Pendleton, M. Grossman, and J. M. Wilson. 1993. Adenovirus-mediated transfer of the CFTR gene to lung of nonhuman primates: toxicity study. Human Gene Ther. 4: 771-780.

29. McGrory, W. J., Bautista, D. S. and Graham, F. L. 1988. A simple technique for rescue of early region I mutations into infectious human adenovirus type 5. Virology, 163, 614-617, 1988.

30. Bett, A. J., Haddara, W., Prevec, L. and Graham, F. L. 1994. An efficient and flexible system for construction of adenovirus vector with insertions or deletion in early regions 1 and 3. Proc. Natl. Acad. Sci. USA, 91, 8802-8806.

31. Spessot, R., Inchley, K., Hupel, T. M. and Bacchetti, S. 1989. Cloning of the herpes simplex virus ICP4 gene in an adenovirus vector: Effects on adenovirus gene expression and replication. Virology, 168, 378-387.

32. Krougliak, V. and Graham, F. L. 1995. Development of cell lines capable of complementing E1, E4 and protein IX defective adenovirus type 5 mutants. Human Gene Therapy, 6, 1575-1586.

We claim:

1. A DNA molecule useful for generating a recombinant adenoviral vector comprising an Ad5 5′ITR with packaging signal and an Ad5 3′ITR, a reporter or effector gene cassette and Ad5 sequence.

2. A DNA molecule of claim 1 wherein said reporter gene cassette is the CMV-EGFP cassette in the opposite orientation as said Ad5 5′ITR.

3. A DNA molecule of claim 1 wherein said reporter gene cassette is the CMV-EGFP cassette in the same orientation as said Ad5 5′ITR.

4. A DNA molecule useful for generating a recombinant adenoviral vector comprising an Ad5 5′ITR with packaging signal, a polylinker, and Ad5 sequence.

5. A DNA molecule of claim 4 wherein said polylinker comprises the restriction enzyme sites for XbaI, XhoI, BglII, EcoRV, NotI, SpeI, SalI, ClaI and BamHI.

6. A DNA molecule comprising an Ad5 5′ITR and an Ad5 3′ITR, a polylinker, and Ad5 sequence.

7. A DNA molecule of claim 6 wherein said polylinker comprises the restriction enzyme sites for XhoI, BglII, EcoRV, NotI, SpeI, SalI and ClaI.

8. A DNA molecule of claim 6 wherein said polylinker comprises the restriction enzyme sites for HindII, XhoI, BglII, EcoRV, NotI, SpeI, SalI, and ClaI.

9. A method for generating a recombinant adenoviral particle using a shuttle vector selected from the group consisting of GT4117, GT4121, GT4142, and GT4141 consisting of the steps of, in combination:
mixing at room temperature one of said shuttle vectors with a helper plasmid;
incubating the mixture at room temperature;
combining said mixture with a suitable transfection preparation;
applying said mixture in said transfection preparation to a 293 cell;
incubating said 293 cell for a sufficient period of time such that adenoviral particles are generated; and,
purifying said recombinant adenoviral particles.

10. A method for generating a infectious, replication-deficient, recombinant adenoviral particle consisting of the steps of, in combination:
mixing at a temperature from 35° C. to 80° C. a shuttle vector and a helper plasmid;
combining said mixture with a suitable transfection preparation;
applying said mixture in said transfection preparation to a 293 cell;
incubating said 293 cell for a sufficient period of time such that an adenoviral particle is generated; and,
purifying said recombinant adenoviral particle whereby an infectious, replication-deficient recombinant adenoviral vector is generated.
